(19) United States
(12) Patent Application Publication
(10) Pub. No.: US 2024/0228455 A1
(43) Pub. Date: Jul. 11, 2024

(54) SYNTHESIS OF CBN AND CBNV

(71) Applicant: TEEWINOT LIFE SCIENCES CORPORATION, New York, NY (US)

(72) Inventor: Malcolm J. Kavarana, Fairfax, VA (US)

(21) Appl. No.: 18/557,449

(22) PCT Filed: Apr. 26, 2022
(86) PCT No.: PCT/US2022/026298

§ 371 Date: Oct. 26, 2023

Related U.S. Application Data
(60) Provisional application No. 63/180,283, filed on Apr. 27, 2021.

(51) Int. Cl. C07D 311/80

(52) U.S. Cl. CPC C07D 311/80

(57) ABSTRACT

The invention includes chemical synthesis for preparing cannabinol (CBN) and cannabinovarin (CBNV). The process is suitable for inter alia the large-scale synthesis of pharmaceutical grade CBN and CBNV.

### CROSS-REFERENCE TO RELATED APPLICATIONS

The present application is a 35 U.S.C. § 371 national phase application from, and claims priority to, International Application No. PCT/US2022/026298, filed Apr. 26, 2022, which claims priority under 35 U.S.C. § 119(e) to U.S. Provisional Application No. 63/180,283, filed Apr. 27, 2021, all of which applications are incorporated herein by reference in their entireties.

### FIELD OF THE INVENTION

The present disclosure relates to a chemical synthesis for producing commercial quantities of pharmaceutical grade cannabinol (CBN) and cannabinovarin (CBNV). CBN is made by the chemical oxidation of cannabidiol (CBD), while CBNV is made by chemical oxidation of CBDV. Also described are therapeutic formulations of CBN and CBNV, their pharmaceutical applications, and their use in the nutraceutical and cosmetic industries.

### BACKGROUND OF THE INVENTION

Cannabinoids are terpenophenolic compounds found in Cannabis sativa, an annual plant belonging to the Cannabaceae family. The plant contains more than 400 chemicals and approximately 70 cannabinoids, which accumulate mainly in the glandular trichomes. Cannabinoids have proven therapeutic potential. For example, cannabidiol (CBD) is a potent antioxidant and anti-inflammatory compound and may provide protection against acute and chronic neuro-degeneration. CBD may also have anti-depressant activity. Another cannabinoid Cannabichromene (CBC) possesses anti-inflammatory, anti-fungal, and anti-viral properties. Thus, cannabinoids are considered promising therapeutics for the treatment of various diseases.

C. sativa produces and stores cannabinoids in an extracellular space called the trichome. The plant produces cannabinoids by contacting a CBGA substrate or a CBGVA substrate with a cannabinoid synthase enzyme. These two substrates are produced in the plant by aromatic prenyltransferase enzymes. For example, prenylation of olivetic acid produces CBGA while prenylation of divariolic acid produces CBGVA.

The cannabinoid synthase enzymes, THCA synthase, CBCA synthase, and CBDA synthase, convert CBGA or CBGVA to primary cannabinoid products THCA, CBCA, CBDA or THCVA, CBCVA, and CBDVA, respectively. Secondary cannabinoid products such as CBN or CBNV are formed by the oxidation of THCA or THCVA, respectively by exposure of the C. sativa plant tissue to sunlight, air, and heat. However, the formation of such secondary cannabinoid products in plants occurs in low amounts. Thus, obtaining large quantities of CBN or CBNV from plant tissue for commercial use is both impractical and cost prohibitive.

Adams, R. et al., J. Am. Chem. Soc., 63:2209-2213 (1941) describe preparation of tetrahydrocannabinol. Isomerization of cannabidiol with para-toluene sulfonic acid (pTsOH) is described. Pollastro, F. et al., J. Nat. Sci., 81:630-633 (2018) propose iodine-promoted aromatization of p-menthane-type phytocannabinoids to provide access to CBN. Further, WO 2020/031179 to Jagtap, P. and Musa, S. (Beetlebung Pharma Ltd.) describes methods for synthesis of cannabinoid compounds.

There remains a need for improved methods to produce CBN and CBNV inter alia, and for effective compositions and uses thereof.

### SUMMARY OF THE INVENTION

In some aspects, the present invention provides chemical synthesis of CBN and CBNV. In some embodiments, methods relate to the chemical oxidation of CBD to CBN. In other embodiments, a similar chemical process is used for the chemical oxidation of CBDV to CBNV. In other embodiments, the described methods are scalable and can be used to produce large quantities of pharmaceutical grade CBN or CBNV.

In other aspects, the present invention provides a process for chemically synthesizing CBN. In one embodiment, the process comprises oxidizing CBD to CBN, comprising:
(a) contacting cannabidiol (CBD) with a solvent and heating the resulting first mixture;
(b) contacting an oxidant with a solvent and heating the resulting second mixture; and
(c) adding the heated mixture of step (b) to the heated mixture of step (a).

In one embodiment, the solvent used for step (a) or step (b) is selected from the group consisting of hexane, heptane, xylene, ortho-xylene, meta-xylene, para-xylene, and toluene.

In another embodiment, the solvent for step (a) or step (b) is xylene or toluene. According to one embodiment, the crude CBN can further be purified. In one embodiment, purification of CBN is carried out by silica gel chromatography, short plug silica gel chromatography, normal phase or reverse phase high performance liquid chromatography (HPLC), or centrifugal partitioning chromatography (CPC) chromatography and combinations thereof.

In one embodiment, the CBN can be purified using a short plug silica gel chromatography, centrifugal partitioning chromatography (CPC) chromatography, or a combination of short plug silica gel chromatography and CPC.

In another embodiment, the first mixture is heated to a temperature in the range from 110° C. to 130° C. In one embodiment, the first mixture comprising CBD and the base is heated to a temperature of 110° C.

In one embodiment, the solvent used to dissolve the oxidant is selected from the group consisting of hexane, heptane, xylene, ortho-xylene, meta-xylene, para-xylene, and toluene. In one embodiment, the solvent used to dissolve the oxidant is xylene or toluene and the temperature of the second mixture comprising the oxidant is in the range from 60° C. to 90° C.

In other embodiments, the temperature of the second mixture is about 60° C.

In some embodiments, the oxidant is selected from the group consisting of bromine, iodine, DDQ, and DDQ/para-toluenesulfonic (p-TsOH) acid mixture and combinations thereof. In one embodiment, the oxidant is iodine (I₂) and the conversion of CBD to CBN occurs in the presence of a base such as an organic base or an inorganic base.

In one embodiment, the base is an inorganic base selected from the group consisting of sodium carbonate, potassium carbonate, sodium bicarbonate, potassium bicarbonate, sodium hydrogen phosphate, and potassium hydrogen phosphate.

In another embodiment, the CBN can be further purified prior to use, by use of a short silica plug or using centrifugal partitioning chromatography (CPC).

In another embodiment and optionally in conjunction with other embodiments, a toluene solution of iodine at 60° C. is contacted with a toluene solution of CBD and base at about 110° C.

In other aspects, the present invention provides a method for oxidizing CBDV to CBNV, the method comprising:
(a) contacting cannabidiol (CBDV) with a solvent and heating the resulting first mixture;
(b) contacting an oxidant with a solvent and heating the resulting second mixture; and
(c) adding the heated mixture of step (b) to the heated mixture of step (a).

In one embodiment, the solvent for step (a) or step (b) is selected from the group consisting of hexane, heptane, xylene, ortho-xylene, meta-xylene, para-xylene, and toluene. According to an embodiment, the solvent is xylene or toluene.

In an embodiment, the first mixture comprising CBDV and the base is heated to a temperature in the range from 110° C. to 130° C. In one embodiment the temperature of the first mixture is 110° C.

In one embodiment, the solvent used to dissolve the oxidant is xylene or toluene and the temperature of the second mixture comprising the oxidant is in a range from 60° C. to 90° C. In an embodiment, the temperature of the second mixture is 60° C.

In other embodiments, the oxidant is selected from the group consisting of bromine, iodine, DDQ, DDQ/para-toluenesulfonic (p-TsOH) acid mixture and combinations thereof. In one embodiment, the oxidant is iodine (I₂) and the conversion of CBDV to CBNV occurs in the presence of a base such as an organic base or an inorganic base.

In one embodiment, the base is an inorganic base selected from the group consisting of sodium carbonate, potassium carbonate, sodium bicarbonate, potassium bicarbonate, sodium hydrogen phosphate, and potassium hydrogen phosphate.

In other aspects, a cannabinovarin (CBNV) produced by the disclosed processes can be further purified using silica gel chromatography, a short plug silica gel chromatography, or centrifugal partitioning chromatography (CPC) prior to use.

In another aspect, the present invention provides a method for synthesizing cannabinol (CBN) or cannabinovarin (CBNV), the method comprising contacting a compound according to Formula 1 with a solvent and an acid to form a first mixture.

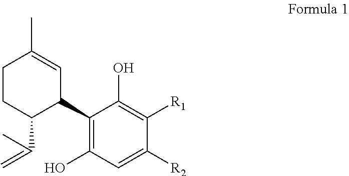

The resulting first mixture is heated to form a compound according to Formula 2; and

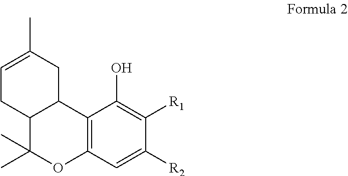

The mixture containing the Formula 2 compound is then contacted with an oxidant and heated to get CBN or CBNV.

For Formula 1 and Formula 2 compounds R₁ is —H and R₂ is either propyl or pentyl.

### BRIEF DESCRIPTION OF THE DRAWINGS

FIG. 1 shows a schematic of a chemical process for synthesizing CBN or CBNV using an inorganic base and an oxidant.

FIG. 2 shows a schematic of a chemical process for synthesizing CBN or CBNV using an acid and an oxidant.

FIG. 3 shows the 1H-NMR spectrum of CBN (CDCl₃-TMS; purity >95%).

FIG. 4 shows HPLC for the synthesis of CBN using an aqueous-organic solvent.

FIG. 5 shows HPLC of crude reaction mixture—CBN synthesis 50 g scale, 11 h.

FIG. 6 shows HPLC of pure CBN synthesis 50 g scale, 11 h.

FIG. 7 shows HPLC, CBD to delta-8 THC—Synthesis 250 g scale, 1.0 h.

FIG. 8 shows delta-8 THC to CBN—Synthesis 250 g scale, 20.0 h at 100 C.

FIG. 9 shows HPLC: CBN Crystals—250 g scale reaction.

FIG. 10 shows Scheme 1.

### DETAILED DESCRIPTION

### Definitions

As used herein, unless otherwise stated, the singular forms “a,” “an,” and “the” include plural reference. Thus, for example, a reference to “a cell” includes a plurality of cells, and a reference to “a molecule” is a reference to one or more molecules.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

The term “alkyl” refers to a straight or branched chain, saturated hydrocarbon having the indicated number of carbon atoms. For example, (C₁-C₁₀)alkyl is meant to include but is not limited to methyl, ethyl, propyl, isopropyl, butyl, sec-butyl, tert-butyl, pentyl, isopentyl, neopentyl, hexyl, isohexyl, and neohexyl, etc. An alkyl group can be unsubstituted or optionally substituted with one or more substituents as described herein below.

The term “alkenyl” or “alkene” refers to a straight or branched chain unsaturated hydrocarbon having the indicated number of carbon atoms and at least one double bond. Examples of a (C₂-C₁₀)alkenyl group include, but are not limited to, ethene, propene, 1-butene, 2-butene, isobutylene, sec-butylene, 1-pentene, 2-pentene, isopentene, 1-hexene, 2-hexene, 3-hexene, isohexene, 1-heptene, 2-heptene, 3-heptene, isoheptene, 1-octene, 2-octene, 3-octene, 4-octene, and isooctene. An alkenyl group can be unsubstituted or optionally substituted with one or more substituents as described herein below.

The term “alkoxy.” refers to an —O-alkyl group having the indicated number of carbon atoms. For example, a (C₁-C₆)alkoxy group includes —O-methyl, —O-ethyl, —O-propyl, —O-isopropyl, —O-butyl, —O-sec-butyl, —O-tert-butyl, —O-pentyl, —O-isopentyl, —O-neopentyl, —O-hexyl, —O-isohexyl, and —O-neohexyl.

The term “aryl” refers to a 3- to 14-member monocyclic, bicyclic, tricyclic, or polycyclic aromatic hydrocarbon ring system. Examples of an aryl group include naphthyl, pyrenyl, and anthracyl. An aryl group can be unsubstituted or optionally substituted with one or more substituents as described herein below.

The terms “alkylene,” “cycloalkylene,” “alkenylene,” “alkynylene,” “arylene,” and “heteroarylene,” alone or as part of another substituent, means a divalent radical derived from an alkyl, cycloalkyl, alkenyl, alkynyl, aryl, or heteroaryl group, respectively, as exemplified by —CH₂CH₂CH₂CH₂—. For alkylene, alkenylene, or aryl linking groups, no orientation of the linking group is implied.

The term “halogen” and “halo” refers to —F, —Cl, —Br or —I.

The term “heteroatom” includes oxygen (O), nitrogen (N), and sulfur (S).

A “hydroxyl” or “hydroxy” refers to an —OH group.

The term “hydroxyalkyl,” refers to an alkyl group having the indicated number of carbon atoms wherein one or more of the alkyl group's hydrogen atoms is replaced with an —OH group. Examples of hydroxyalkyl groups include, but are not limited to, —CH₂OH, —CH₂CH₂OH, —CH₂CH₂CH₂OH, —CH₂CH₂CH₂CH₂OH, —CH₂CH₂CH₂CH₂CH₂OH, —CH₂CH₂CH₂CH₂CH₂CH₂OH, and branched versions thereof.

The term “cycloalkyl” or “carbocycle” refer to monocyclic, bicyclic, tricyclic, or polycyclic, 3- to 14-membered ring systems, which are saturated, unsaturated or aromatic. Representative examples of cycloalkyl include, but are not limited to cyclopropyl, cyclobutyl, cyclopentyl, cyclohexyl, naphthyl, anthracyl, phenyl, benzofuranyl, and benzothiophenyl. A cycloalkyl group can be unsubstituted or optionally substituted with one or more substituents as described herein below.

The term “cycloalkene” refers to monocyclic, bicyclic, tricyclic, or polycyclic, 3- to 14-membered ring systems, which are unsaturated, that is comprising 1 or more double bonds. Exemplary cycloalkenes include cyclopropene, cyclobutene, cyclopentene, and cyclohexene. A cycloalkene group can be unsubstituted or optionally substituted with one or more substituents as described herein below.

The terms “oxidant” or “oxidizing agent” refer to a compound, reagent, or reactant that removes electrons from another reactant during a redox reaction. The oxidizing agent or oxidant typically accepts these electrons (gains electrons) and is reduced. An oxidizing agent is an electron acceptor. In one embodiment, an oxidizing agent may also be viewed as a species capable of transferring electronegative atoms (especially oxygen) to a substrate. Examples of oxidizing agents include without limitation, potassium permanganate, oxygen, hydrogen peroxide, a halogen (e.g., chlorine, bromine, iodine), an alkali metal nitrate, as well as other reagents commonly known in the chemical art.

The term “amine or amino” refers to an —NR_{c}R_{d} group wherein Re and Ra each independently refer to a hydrogen, (C₁-C₅)alkyl, aryl, (C₁-C₅)haloalkyl, and (C₁-C₆)hydroxyalkyl group.

The terms “carboxyl” and “carboxylate” include such moieties as may be represented by the general formula:

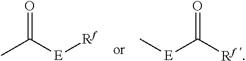

E in the formula is a bond or O and R^{f} individually is H, alkyl, alkenyl, aryl, or a pharmaceutically acceptable salt. Where E is O, and R^{f} is as defined above, the moiety is referred to herein as a carboxyl group, and particularly when R^{f} is a hydrogen, the formula represents a “carboxylic acid”. In general, where the expressly shown oxygen is replaced by sulfur, the formula represents a “thiocarbonyl” group.

If there is a discrepancy between a depicted structure and a name given that structure, then the depicted structure controls.

In the context of the present disclosure the term “analog” refers to a compound that is structurally related to naturally occurring cannabinoids, but whose chemical and biological properties may differ from naturally occurring cannabinoids. In the present context, analog or analogs refer compounds that may not exhibit one or more unwanted side effects of a naturally occurring cannabinoid. Analog also refers to a compound that is derived from a naturally occurring cannabinoid by chemical, biological or a semi-synthetic transformation of the naturally occurring cannabinoid.

The term “prodrug” refers to a precursor of a biologically active pharmaceutical agent (drug). Prodrugs must undergo a chemical or a metabolic conversion to become a biologically active pharmaceutical agent. A prodrug can be converted ex vivo to the biologically active pharmaceutical agent by chemical transformative processes. In vivo, a prodrug is converted to the biologically active pharmaceutical agent by the action of a metabolic process, an enzymatic process or a degradation that removes the prodrug moiety to form the biologically active pharmaceutical agent.

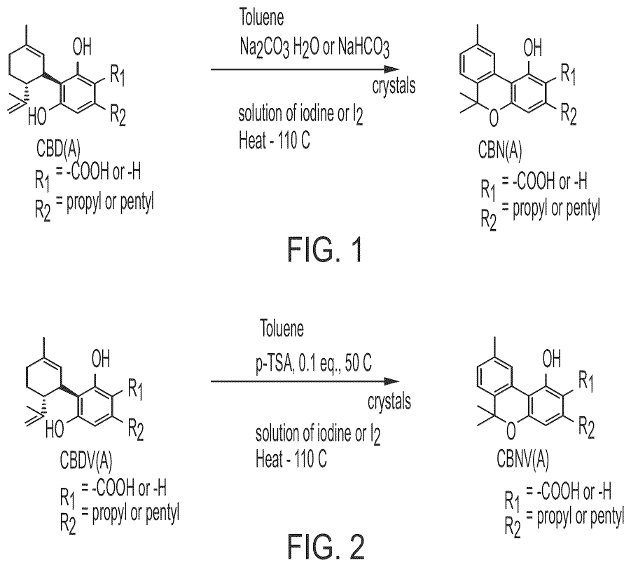

In one of its embodiments the present disclosure provides methods for synthesizing CBN and CBNV, respectively. Overall, the methods comprise the step of oxidizing CBD or CBDV to CBN or CBNV respectively, following an in-situ cyclization of CBD or CBDV to THC or THCV, respectively. The THC or THCV produced are not isolated and undergo rapid oxidation to CBN, and CBNV, respectively. See FIGS. 1 and 2 that structurally depict the conversion of CBD and CBDV to CBN and CBNV, respectively.

The CBN or CBNV thus obtained are purified using various chromatographic methods, for example, using silica gel chromatography, reverse-phase silica chromatography, centrifugal portioning chromatography (CPC), high pressure liquid chromatography (HPLC), or via solvent-antisolvent precipitation. The purity of CBN or CBNV is verified using HPLC. The purity of CBN or CBNV is in the range from about 90% to about 99.8%, about 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99%, 99.5%, or 99.8%.

In one embodiment the purity of CBN or CBNV is in the range from about 95% to about 99.5%, about 96% to about 99.5%, about 97% to about 99.5%, about 98% to about 99.5%, or about 99% to about 99.5%. In one embodiment, the purified CBN or CBNV can be used as pharmaceutical agents.

In one embodiment, the conversion of CBD or CBDV to CBN or CBNV, respectively, occurs in the presence of a base. Both organic and inorganic bases can be used in the claimed process. In some embodiments, an organic base is used. In other embodiments, an inorganic base is used. Suitable organic bases include alkyl amines with general formulae R₃NH₂ or (R₃)(R₄)NH, cycloalkyl amines, or aryl amines with general formulae R₅NH₂, or (R₅)(R₆)NH. Substituents R₃ and R₄ are each independently (C₁-C₈) linear of branched alkyls, such as methyl, ethyl, propyl, iso-propyl, butyl, sec-butyl, tert-butyl, pentyl, hexyl, heptyl, octyl groups or combinations thereof. In one embodiment, substituents R₃ and R₄ together with the —NH group form a cycloalkyl amine ring system. Substituents R₅ and R₆ are each independently phenyl, substituted phenyl, e.g., a phenyl group substituted at positions 2-, 3-, or 4- with a methyl group, cyclobutyl, cyclopentyl, cyclohexyl groups. Exemplary substituents on the phenyl ring of aromatic amines include nitro, cyano, halogen, hydroxyl, and —OR₇ groups, where R₇ is a (C₁-C₄) alkyl. Some exemplary alkyl and aromatic amines include di-isopropyl ethylamine (DIPEA), methylamine, ethylamine, tert-butylamine, bis-tert-butylamine, aniline, N-methyl aniline, 4-methoxyaniline, pyridine, 4-piperidinopyridine, piperidine, 4-methylpiperidine, N-methyl-2-pyrrolidone, or N-phenylpiperidine.

The conversion of CBD or CBDV to CBN or CBNV, respectively, can also occur in the presence of an inorganic base. Suitable inorganic bases include without limitation alkali and alkaline earth metal hydroxides, alkali and alkaline earth metal carbonates, or alkali and alkaline earth metal bicarbonates. Exemplary inorganic bases include lithium carbonate, sodium carbonate, potassium carbonate, magnesium carbonate, calcium carbonate, ammonium carbonate, sodium bicarbonate, potassium bicarbonate, ammonia, ammonium hydroxide, lithium hydroxide, sodium hydroxide, potassium hydroxide, magnesium hydroxide, calcium hydroxide, and strontium hydroxide.

In one embodiment, the conversion of CBD or CBDV to CBN or CBNV respectively, is carried out in the presence of an acid. The acid can be a Lewis acid or a protic acid. Exemplary acids are hydrochloric acid, para-toluene sulfonic acid (p-TsOH), citric acid, aluminum trichloride, boron trifluoride etherate, and magnesium sulfate. In some embodiments, the acid is resin bound. Exemplary resin bound acids include strongly acidic or weakly acidic ion exchange resins that have sulfonic acid or carboxylic acid groups. Commercially available sulfonic acid resins include without limitation, Amberlite or Amberlyst IR-120 Plus (H), IR-120 Plus, A-15, IRP-69, and 1200(H) resins. Other commercially available sulfonic acid-based ion exchange resins are the Dowex family of resins, for example, 50WX2-100, 50WX2-200, 50WX2-400, 50WX4-50, 50WX4-100, 50WX4-200, 50WX4-200(R), and 50WX4-400. Weakly acidic ion exchange resins that are suitable for the conversion of CBD(V) to CBN(V) respectively, include carboxylic acid-based ion-exchange resins, such as Amberlite CG-50 (Type 1), Amberlite IRC-50, Amberlite IRC 50S, Amberlite IRP64, and Dowex CCR-3.

In one embodiment, the acid used is p-TSA, or Amberlite (Amberlyst) A-15. According to one embodiment, the acid is Amberlite CG-50 (Type 1), Amberlite IRC-50, or Amberlite IRC 50S. In one embodiment, the acid is Amberlite CG-50 (Type 1), Amberlite IRC-50, or Amberlite IRC 50S. In one embodiment, the acid is Amberlite IRP64, and Dowex CCR-3.

In one embodiment, the acid is p-TSA. In one embodiment, the acid is Amberlite (Amberlyst) A-15.

In one of its embodiments the acid promotes the in-situ cyclization of CBD or CBDV to delta-8-THC or delta-8 THCV, respectively followed by in-situ aromatization (oxidation) of delta-8-THC or delta-8 THCV to CBN or CBNV respectively, that is, the delta-8-THC or delta-8 THCV produced are not isolated and undergo rapid oxidation to CBN, and CBNV, respectively.

### Compositions

CBN or CBNV synthesized using the presently disclosed methods are administered to a patient or subject in need of treatment either alone or in combination with other compounds having similar or different biological activities.

For example, CBN or CBNV and a composition comprising these compounds can be administered in a combination therapy routine, i.e., either simultaneously in single or separate dosage forms or in separate dosage forms within hours or days of other compounds having similar biological activities. Examples of such combination therapies include administering a composition comprising CBN or CBNV with other pharmaceutical agents used to treat glaucoma. Other examples of therapeutic use include compositions of CBN or CBNV for the treatment of pain including neuropathic pain, for the treatment of microbial infection, for the treatment of inflammation, for the treatment of cancer, for treatment related to improving bone health, for use as a sedative, and use as an appetite stimulant.

The present disclosure also provides a pharmaceutical composition comprising a pharmaceutically acceptable salt or solvate of CBN or CBNV in admixture with a pharmaceutically acceptable carrier. In some embodiments, the composition further contains, in accordance with accepted practices of pharmaceutical compounding, one or more additional therapeutic agents, pharmaceutically acceptable excipients, diluents, adjuvants, stabilizers, emulsifiers, preservatives, colorants, buffers, flavor imparting agents.

The presently disclosed compositions can be administered orally, topically, parenterally, by inhalation or spray in dosage unit formulations. The term parenteral as used herein includes subcutaneous injections, intravenous, intramuscular, intrasternal injection or infusion techniques.

Suitable oral compositions in accordance with the present disclosure include without limitation tablets, troches, lozenges, aqueous or oily suspensions, dispersible powders or granules, emulsion, hard or soft capsules, syrups or elixirs.

Compositions suitable for oral use may be prepared according to any method known to the art for the manufacture of pharmaceutical compositions. For instance, liquid formulations of CBN or CBNV contain one or more agents selected from the group consisting of sweetening agents, flavoring agents, coloring agents and preserving agents in order to provide pharmaceutically elegant and palatable preparations of CBN or CBNV.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

For aqueous suspensions, the CBN or CBNV is admixed with excipients suitable for maintaining a stable suspension. Examples of such excipients include without limitation are sodium carboxymethylcellulose, methylcellulose, hydropropylmethylcellulose, sodium alginate, polyvinylpyrrolidone, gum tragacanth and gum acacia.

Oral suspensions can also contain dispersing or wetting agents, such as naturally occurring phosphatide, for example, lecithin, polyoxyethylene stearate, heptadecaethyleneoxycetanol, polyoxyethylene sorbitol monooleate, polyethylene sorbitan monooleate. The aqueous suspensions may also contain one or more preservatives, for example ethyl, or n-propyl p-hydroxy benzoate, one or more coloring agents, one or more flavoring agents, and one or more sweetening agents, such as sucrose or saccharin.

Syrups and elixirs may be formulated with sweetening agents, for example glycerol, propylene glycol, sorbitol or sucrose. Such formulations may also contain a demulcent, a preservative, and flavoring and coloring agents. The pharmaceutical compositions may be in the form of a sterile injectable, or an aqueous suspension. This suspension may be formulated according to the known art using those suitable dispersing or wetting agents and suspending agents. The sterile injectable preparation may also be sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. In addition, fatty acids such as oleic acid find use in the preparation of injectables.

Compositions for parenteral administrations are administered in a sterile medium. Depending on the vehicle used and concentration the concentration of the drug in the formulation, the parenteral formulation can either be a suspension or a solution containing dissolved drug. Adjuvants such as local anesthetics, preservatives and buffering agents can also be added to parenteral compositions.

The total amount by weight of CBN or CBNV in a pharmaceutical composition is from about 0.1% to about 95%. By way of illustration, the amount of CBN or CBNV by weight of the pharmaceutical composition can be about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 2.1%, about 2.2%, about 2.3%, about 2.4%, about 2.5%, about 2.6%, about 2.7%, about 2.8%, about 2.9%, about 3%, about 3.1%, about 3.2%, about 3.3%, about 3.4%, about 3.5%, about 3.6%, about 3.7%, about 3.8%, about 3.9%, about 4%, about 4.1%, about 4.2%, about 4.3%, about 4.4%, about 4.5%, about 4.6%, about 4.7%, about 4.8%, about 4.9%, about 5%, about 5.1%, about 5.2%, about 5.3%, about 5.4%, about 5.5%, about 5.6%, about 5.7%, about 5.8%, about 5.9%, about 6%, about 6.1%, about 6.2%, about 6.3%, about 6.4%, about 6.5%, about 6.6%, about 6.7%, about 6.8%, about 6.9%, about 7%, about 7.1%, about 7.2%, about 7.3%, about 7.4%, about 7.5%, about 7.6%, about 7.7%, about 7.8%, about 7.9%, about 8%, about 8.1%, about 8.2%, about 8.3%, about 8.4%, about 8.5%, about 8.6%, about 8.7%, about 8.8%, about 8.9%, about 9%, about 9.1%, about 9.2%, about 9.3%, about 9.4%, about 9.5%, about 9.6%, about 9.7%, about 9.8%, about 9.9%, about 10%, about 11%, about 12%, about 13%, about 14%, about 15%, about 16%, about 17%, about 18%, about 19%, about 20%, about 25%, about 30%, about 35%, about 40%, about 45%, about 50%, about 55%, about 60%, about 65%, about 70%, about 75%, about 80%, about 85%, about 90% or about 95%.

In one embodiment, the pharmaceutical composition comprises a total amount by weight of CBN or CBNV of about 1% to about 10%; about 2% to about 10%; about 3% to about 10%; about 4% to about 10%; about 5% to about 10%; about 6% to about 10%; about 7% to about 10%; about 8% to about 10%; about 9% to about 10%; about 1% to about 9%; about 2% to about 9%; about 3% to about 9%; about 4% to about 9%; about 5% to about 9%; about 6% to about 9%; about 7% to about 9%; about 8% to about 9%; about 1% to about 8%; about 2% to about 8%; about 3% to about 8%; about 4% to about 8%; about 5% to about 8%; about 6% to about 8%; about 7% to about 8%; about 1% to about 7%; about 2% to about 7%; about 3% to about 7%; about 4% to about 7%; about 5% to about 7%; about 6% to about 7%; about 1% to about 6%; about 2% to about 6%; about 3% to about 6%; about 4% to about 6%; about 5% to about 6%; about 1% to about 5%; about 2% to about 5%; about 3% to about 5%; about 4% to about 5%; about 1% to about 4%; about 2% to about 4%; about 3% to about 4%; about 1% to about 3%; about 2% to about 3%; or about 1% to about 2%.

### EXAMPLES

### Example 1

### General Protocol for the Synthesis of CBN or CBNV Using a Base and an Oxidant

The conversion of CBD to CBN or CBDV to CBNV, was initiated by dissolving either CBD or CBDV (substrate) in a solvent like xylene or toluene at a dilution ratio of substrate:solvent in the range from 1:30-1:200. The mixture was stirred mechanically at room temperature until the substrate completely dissolved. To this solution was added a base and the solution was flushed with nitrogen for about 30 minutes (Solution 1). Solution 1 was gradually heated under an inert atmosphere, until the temperature of the solution was in a range from about 85° C. to 130° C.

In a separate container, the oxidant, for example iodine, or P₂O₅/hexamethyldisiloxane/SeO₂, or bromine was contacted with a solvent like xylene or toluene at a oxidant:solvent dilution ratio of 1:10-1:30. The mixture was stirred mechanically at room temperature, then flushed with an inert gas such as nitrogen, and heated while maintaining the inert atmosphere to a temperature in the range from about 60° C.-90° C. (Solution 2).

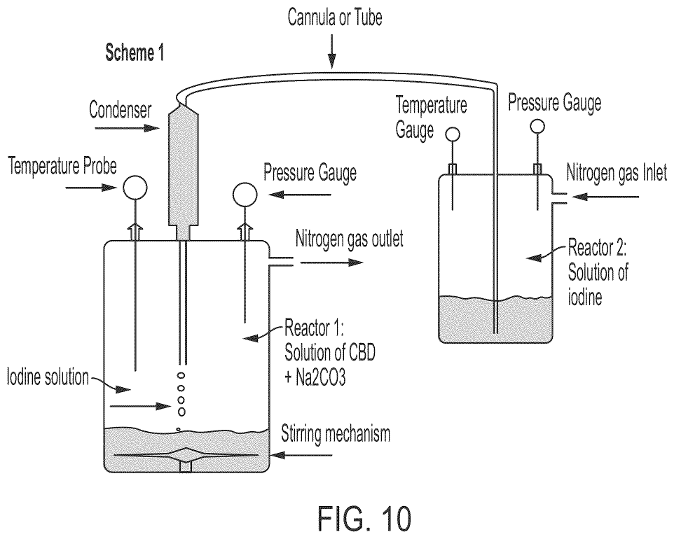

Solution 2, was then gradually added to Solution 1 (hot solution of CBD or CBDV and base), over a period of 40-90 minutes. See FIG. 10 (Scheme 1). The addition of Solution 2 to Solution 1 was carried out at a rate that maintained the temperature of Solution 1 in a range from about 85° C. to 130° C., for example, in a range of about 100° C.-130° C., 105° C.-130° C., in a range of about 110° C.-130° C., in a range of about 115° C.-130° C., in a range of about 120° C.-130° C., or in a range of about 125° C.-130° C. After the addition of Solution 2 to Solution 1 was completed, the reaction mixture was stirred under an inert atmosphere while maintaining the temperature of the reaction mixture in a range of about 85° C.-130° C. Progress of the reaction, namely, the conversion of CBD to CBN or CBDV to CBNV, was monitored using thin layer chromatography (TLC) or high-performance liquid chromatography (HPLC) by sampling an aliquot of the reaction mixture at different intervals of time.

Alternatively, solid iodine crystals were directly added to the hot solution of CBD or CBDV and base. The reaction mixture was stirred under an inert atmosphere while maintaining the temperature of the reaction mixture in a range of about 85° C.-130° C. Progress of the reaction, namely, the conversion of CBD to CBN or CBDV to CBNV, was monitored using thin layer chromatography (TLC) or high-performance liquid chromatography (HPLC) by sampling an aliquot of the reaction mixture at different intervals of time.

The reaction was stopped when greater than 90%-95% of the substrates CBD or CBDV were converted to CBN or CBNV, respectively, based on TLC or HPLC analysis. The reaction mixture was cooled to room temperature. The sodium carbonate was filtered off and the filtrate washed with an aqueous solution of citric acid (citric acid:water—1:20 ratio; or 10% citric acid by weight). Alternatively, the cooled reaction mixture containing sodium carbonate was directly quenched using an aqueous solution of 10% to 25% citric acid. After the evolution of bubbles ceased, the organic layer containing crude product was separated from the aqueous layer. The pH of the aqueous layer was measured and a second quench (extraction) with aqueous citric was carried out if the pH of the reaction mixture was above 3.5.

The organic layer containing the crude CBN product or CBNV product was extracted with an aqueous solution of sodium sulfite (Na₂SO₃) or sodium thiosulfate (Na₂S₂O₃) to neutralize unreacted oxidant. Neutralization of oxidant, such as iodine, was ascertained using starch paper. Following extraction with sodium thiosulfate or sodium sulfite, the organic layer was washed with water, dried using anhydrous sodium sulfate and concentrated using a rotary evaporation or via distillation. The crude products, namely CBN or CBNV, were purified as further described below.

### Example 2

### General Protocol for the Conversion of CBD or CBDV to Delta-8-THC or Delta-8 THCV, Respectively, Using an Acid and the Oxidation of Delta-8-THC or Delta-8 THCV to CBN or CBNV

To a solution of CBD or CBDV in a solvent like xylene or toluene (dilution ratio substrate:solvent in the range from 1:10-1:100), was added 0.1 equivalents to 2.0 equivalents of an acid. The solution was heated to a temperature in the range from about 50° C. to 110° C. and maintained at this temperature until thin-layer chromatography (TLC) or high-pressure liquid chromatography (HPLC) showed quantitative conversion of CBD to delta-8 THC or CBDV to delta-8 THCV. The structure of delta-8 THC was confirmed by 1H-NMR.

The reaction mixture containing delta-8 THC was cooled to room temperature. A solution of iodine in toluene or xylene was then added to the reaction mixture, followed by the addition of a base, such as solid Na₂CO₃·H₂O or NaHCO₃. The flask containing the reaction mixture was fitted with a condenser and heated to a temperature in the range from 80° C. to 110° C. under an inert atmosphere.

In one embodiment, iodine crystals were added to the reaction mixture containing delta-8 THC at room temperature, followed by the addition of a base, such as solid Na₂CO₃·H₂O or NaHCO₃. The flask containing the reaction mixture is fitted with a condenser and heated to a temperature in the range from 80° C. to 110° C. under an inert atmosphere.

The reaction was stopped when greater than 90%-95% of delta-8 THC or delta-8 THCV were converted to CBN or CBNV, respectively, based on TLC or HPLC analysis. The reaction mixture was cooled to room temperature. The organic layer containing the crude CBN product or CBNV product was extracted with an aqueous solution of sodium sulfite (Na₂SO₃) or sodium thiosulfate (Na₂S₂O₃) to neutralize unreacted oxidant. Following extraction with sodium thiosulfate, the organic layer was washed with water, dried using anhydrous sodium sulfate and concentrated using a rotary evaporation or via distillation. The crude product, namely CBN or CBNV, were purified as further described below.

### Example 3

### General Method for Purification of CBN

Purification of CBN was carried out using different methods, including silica gel chromatography, a short plug silica gel chromatography, or centrifugal partitioning chromatography (CPC). Separation of the desired product from impurities was achieved using a Biotage column in one instance. At the 10 gram scale, pure CBN (95% to 99% purity) was obtained by passing the crude reaction mixture through a short silica plug or using CPC.

Short-plug silica column chromatography was performed using 5 weight equivalents of silica (50.0 g of silica at the 10 g scale) for purification of CBN. The silica gel column was made by pouring a slurry, e.g., 50 g of silica in about 100 mL-150 mL of heptane, into a glass or a stainless-steel column with an ‘Aspect Ratio’ of about 2.0. After packing the column, the solvent (heptane) was drained under positive pressure (compressed air or nitrogen) or using a vacuum until the solvent reached the surface of the silica bed.

The concentrated, crude reaction mixture, dissolved in a minimal volume of heptane was loaded onto the silica bed. The crude reaction mixture was permitted to adsorb onto the silica bed by allowing the toluene or heptane solution of the crude reaction (collected as Fraction 1), to exit the column.

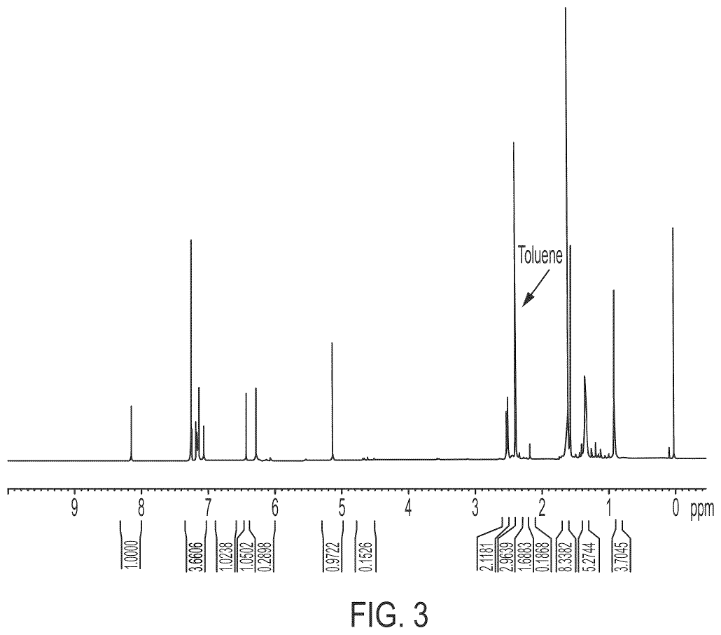

Purification of CBN was carried out by eluting the column with 100% heptane (2×40 mL). The heptane exiting the column was collected as Fractions 2 and 3 (40 mL each) respectively. The solvent composition was modified to contain up to 10% ethyl-acetate in heptane and used to separate CBN from other impurities including unreacted CBD (˜200 mL, combining fractions 4-7, 40-50 mL each). The fractions containing CBN were identified by HPLC or TLC, pooled and concentrated to obtain pure CBN. HPLC analysis of the pooled CBN fractions showed a purity of 95% or greater. The identity of the purified material was confirmed by mass-spectrometry and proton nuclear magnetic resonance (1H-NMR) spectroscopy. See FIG. 3.

In one embodiment, the purity of CBN is in the range from about 80% to 99.9%, for instance in the range from 81% to 99.9%, 82% to 99.9%, 83% to 99.9%, 84% to 99.9%, 85% to 99.9%, 86% to 99.9%, 87% to 99.9%, 88% to 99.9%, 89% to 90.9%, 91% to 99.9%, 92% to 99.9%, 93% to 99.9%, 94% to 99.9%, 95% to 99.9%, 96% to 99.9%, 97% to 99.9%, 98% to 99.9%, or greater than 99.0% by analytical methods known in the chemical art including high performance liquid chromatography (HPLC), gas chromatography (GC), quantitative thin layer chromatography (TLC), quantitative UV-Vis spectroscopy and the like.

### Example 4

### Synthesis of CBN Using an Aqueous-Organic Solvent

A 5 L four neck reactor and 2 L 3-neck round bottom flask were assembled and connected with a ⅜″ poly cannula. To the 5 L reactor was charged CBD (50.0 g, 1.00 equiv), sodium carbonate (50.6 g, 3.0 equiv), water (8.5 mL, 3.0 equiv) and toluene (1.0 L, 20 vols). The 2.0 L 3-neck RBF was charged with iodine (88.8 g, 2.2 equiv) and toluene (500 mL, 10 vols based on CBD). The CBD solution was heated to 100° C. and the iodine solution was warmed to 60° C. The iodine solution was transferred into the 5.0 L reactor over 15 minutes. The flask was rinsed with 50 mL of toluene to transfer all the iodine. Heating was heating continued at 100° C. and the reaction was monitored every two hours by HPLC by preparing standard solutions.

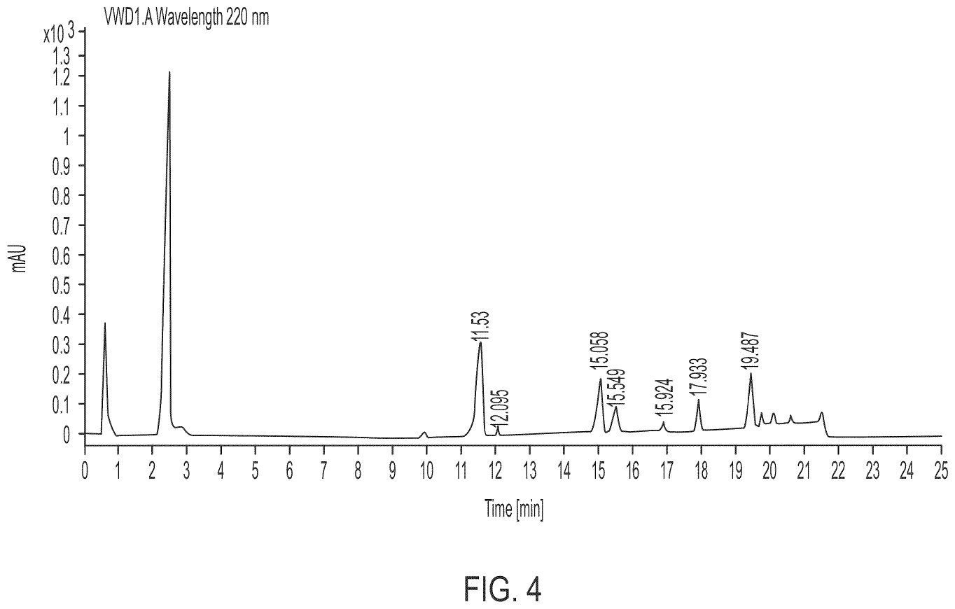

It was observed that in the presence of water a side product is formed which shows a retention time of about 15.0 min (and a mass=618.9 daltons) at about 8 h from the start of the reaction. The reaction mixture was cooled to room temperature and stirred for 16 h. HPLC analysis after stirring the reaction at room temperature for 16 h showed formation of extra impurities. See FIG. 4.

### Example 5

### Small Scale Synthesis of CBN Using a Base

A dry IL 3-neck round bottom flask (RBF) and a dry 250 mL 3-neck RBF were assembled and connected with a ⅛″ poly cannula as shown in Scheme 1 (see FIG. 10). The system was purged with nitrogen for about 30 minutes then evacuated and re-filled with nitrogen. The 1 L flask was charged with CBD (10.0 g, 1.00 equiv.), sodium carbonate monohydrate (11.9 g, 3.0 equiv.), and toluene (300 mL, 30 vols). The 250 mL RBF was charged with iodine (18 g, 2.2 equiv.) and toluene (100 mL, 10 vols based on CBD). The CBD solution was heated to 110° C. and the iodine solution was heated to 60° C.

After the temperatures of both solutions were at the prescribed values (˜20 minutes), the hot iodine solution was cannulated over a period of 10-15 minutes into the 1 L flask containing the solution of CBD and sodium carbonate monohydrate. After transfer of the iodine solution was completed, the reaction mixture was maintained at 110° C. and progress of the reaction was monitored periodically by HPLC or TLC analysis. After about 6.5 hours, HPLC analysis of the reaction showed that >95% of the starting material CBD was converted to desired product CBN. Heating was stopped, and the reaction mixture was cooled to room temperature.

To the crude reaction mixture, at room temperature was filtered, and then extracted with 150 mL of a 10% citric acid solution (30 g citric acid dissolved in 300 mL water, 3.0 equiv. by weight w.r.t CBD) to neutralize any unreacted sodium carbonate monohydrate. After the evolution of carbon dioxide gas ceased, the mixture was transferred to a separatory funnel and the organic layer was extracted. The aqueous and organic layers were permitted to separate, the aqueous layer was removed from the separatory funnel, and the organic layer containing crude CBN was extracted once more using 10% aqueous citric acid (150 mL).

The neutral organic layer was then extracted with an aqueous solution of 10% sodium sulfite (2×150 mL) to remove unreacted excess iodine, followed by a wash with distilled water (2×150 mL). HPLC analysis of the organic layer after aqueous work-up showed >95% CBN by peak area. Purification was carried out by reducing the volume of the toluene solution containing crude CBN to ˜50 mL by rotary evaporation, followed by purification using short plug silica gel chromatography as described in Example 3, or purification by CPC. Fractions containing CBN were pooled, dried and the purity and structural identity of CBN confirmed by HPLC and 1H-NMR spectroscopy. See FIG. 3.

### Example 6

### Synthesis—CBD to delta-8-THC to CBN

### A. Conversion of CBD to Delta-8-THC in the Presence of an Acid

A dry IL 3-neck round bottom flask (RBF) was charged with CBD (10.0 g, 0.0318 moles, 1.00 equiv.), p-TSA (0.55 g, 0.1 equiv.), and toluene (400 mL, 40 vols). The CBD solution was heated to 50° C. and progress of the cyclization reaction, CBD to delta8-THC, was monitored periodically by HPLC or TLC analysis. The reaction mixture was cooled to room temperature when HPLC analysis showed quantitative conversion of CBD to delta-8-THC, approximately 1 hour.

### B. Conversion of Delta-8-THC to CBN

Iodine crystals (17.7 g. 2.2 eq.), and solid NaHCO₃ (10.7 g, 4.0 eq), were then added to the reaction flask. The reaction flask was fitted with a condenser, a temperature probe and heated to 110° C. Progress of the aromatization reaction, delta-8-THC to CBN, was monitored periodically by HPLC or TLC analysis. After about 8.0 hours, HPLC analysis showed that >95% of the starting material delta-8-THC was converted to desired product CBN. Heating was stopped, and the reaction was cooled to room temperature. The reaction mixture was filtered to remove NaHCO₃ and inorganic solids, prior to extractive workup.

To the crude reaction mixture, at room temperature, was added 150 mL of a 10% citric acid solution (30 g citric acid dissolved in 300 mL water, 3.0 equiv. by weight w.r.t CBD) to neutralize any remaining sodium bicarbonate. After the evolution of carbon dioxide gas ceased, the mixture was transferred to a separatory funnel and the organic layer was extracted with 10% aqueous citric acid (150 mL). The aqueous and organic layers were permitted to separate, following which the aqueous layer was removed from the separatory funnel, and the organic layer containing crude CBN was extracted once more using 10% aqueous citric acid (150) mL).

The neutral organic layer was then extracted with an aqueous solution of 10% sodium thiosulfate (2×150 mL) to remove unreacted excess iodine, followed by a wash with distilled water (2×150 mL). HPLC analysis of the organic layer after aqueous work-up showed >95% CBN by peak area. Purification was carried out by reducing the volume of the toluene solution containing crude CBN to ˜50 mL by rotary evaporation, followed by purification using short plug silica gel chromatography, purification by CPC, or crystallization. In one embodiment, a solution of crude CBN (˜16 g) in heptane was loaded onto a silica gel column (300 g) and eluted using a gradient comprising 100% heptane to 2.0% ethyl acetate in heptane. Fractions containing CBN were pooled, dried and the purity and structural identity of CBN confirmed by HPLC and 1H-NMR spectroscopy. See FIG. 3 for a representative ¹H-NMR.

### Example 7

### Synthesis of CBN at 50 Gram Scale

A larger scale synthesis of CBN was carried out in a 5 L reactor attached with a reflux condenser, a stir shaft, and a temperature probe. CBD (50 g, 159 mmol, 1.0 eq.) was charged to the reactor followed by toluene (2.0 L, 40 vol) and pTSA·H₂O (3.0 g, 15.9 mmol, 0.1 equiv.). The reaction mixture was heated to 50° C. and stirred at 50° C. for 1 h. HPLC analysis after 1 h showed the complete conversion of CBD to Δ⁸-THC. The mixture was cooled to 20° C. NaHCO₃(54.8 g, 625.3 mmol, 4.1 equiv.) and iodine crystals (88.8 g, 349.8 mmol, 2.2 equiv.) were then charged into the reactor containing the reaction mixture. The reaction mixture was heated to 110° C. (process temperature 104-105° C.) and the reaction progress followed by HPLC every hour. After 10 h HPLC analysis of the reaction mixture showed >99% conversion of Δ⁸-THC to CBN. The peak corresponding to CBN in the chromatogram showed 91.3% CBN. At this point, heating was stopped and the reaction mixture was cooled to room temperature. The solid NaHCO₃ was filtered and the filtrate containing crude CBN was washed with 10% citric acid solution (336 mL, 1.1 equiv.) and 5% Na₂SO₃ (750 mL, 15 volumes), water (750 mL, 15 volumes). Toluene was removed under reduced pressure and the brown oil (91% pure) was purified by silica plug.

### Purification of CBN

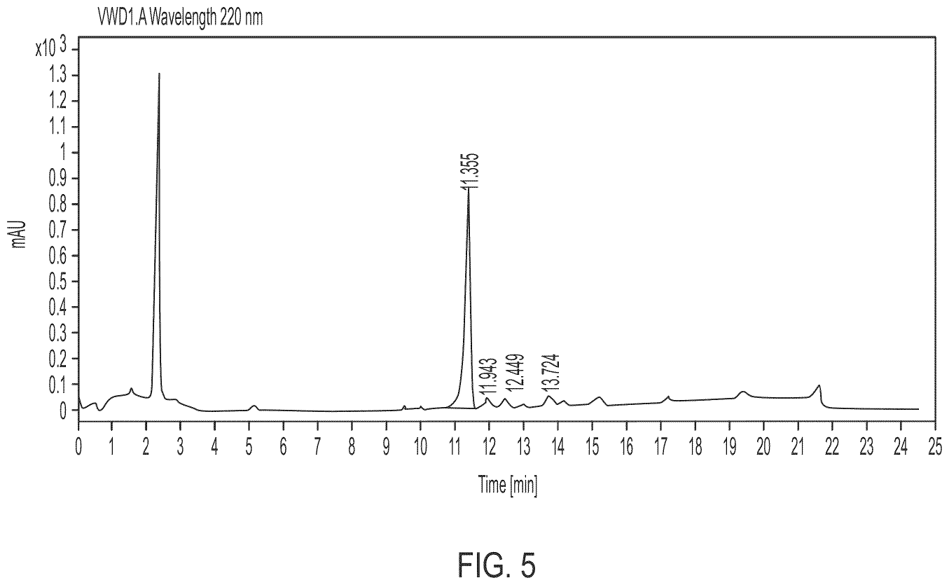

The crude compound was dissolved in heptanes (200 mL) loaded onto a silica plug (750 g, 15 wt, 6″ (L)×5″ (W)) which had been conditioned with heptanes (2 L). The silica plug was eluted using 100% heptane followed by a solvent system comprising from 1.1% ethyl acetate-hexane to 1.5% ethyl acetate-hexane. Column fractions were monitored by TLC and fractions containing CBN were pooled. A second 50.0 g scale synthesis was carried out as described above. The reaction was carried out using a protocol similar to the one described above and conversion of CBD to Δ⁸-THC followed by oxidation of Δ⁸-THC to CBN proceeded as discussed above. The total reaction time for oxidation of Δ⁸-THC to CBN was 11 h. HPLC of the reaction mixture at 11 h showed 99.3% conversion of Δ⁸-THC to CBN. See FIG. 5. The peak corresponding to CBN in the chromatogram showed 92.7% CBN.

The crude CBN was dissolved in heptanes (100 mL) loaded onto a silica plug (500 g, 15 wt, 3′ (h)×5′ (w)) which had been conditioned with heptanes (2 L). The silica plug was eluted using 100% heptane followed by a solvent system comprising from 1.1% ethyl acetate-hexane to 1.5% ethyl acetate-hexane. Column fractions were monitored by TLC and fractions containing CBN were pooled. The purity of the pooled CBN fractions (51.1 g, oil) was ˜92% by HPLC.

### Crystallization

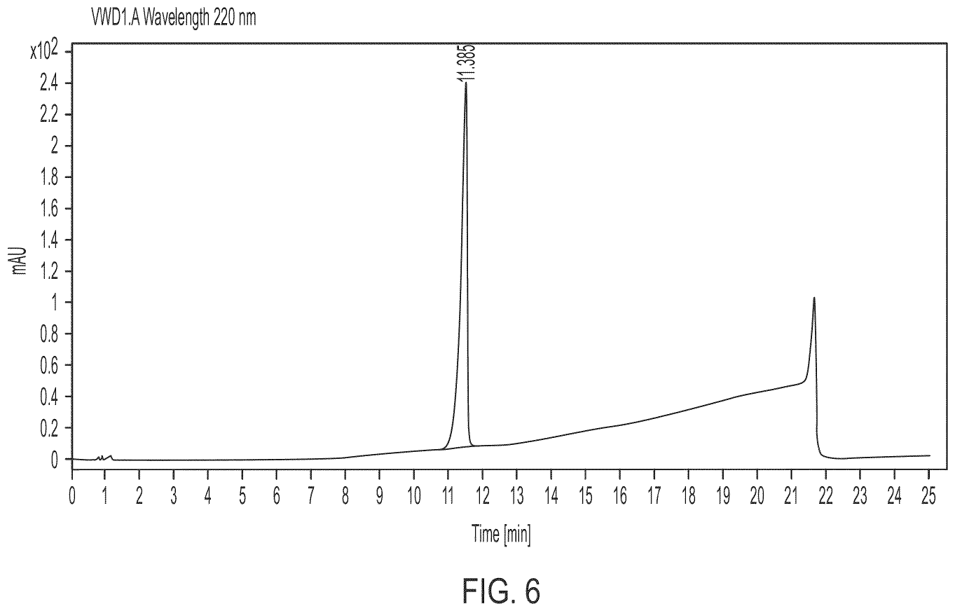

The CBN oil (51.1 g) was redissolved in heptanes (150 mL, 3 volumes) followed by the addition of seed crystals (10 mg) and stored in the freezer overnight for crystallization. The white solid obtained was filtered, washed with heptanes (50 mL, 1 volume) and dried in a vacuum oven to isolate 26.8 g (54% yield) of CBN as a white solid. HPLC=100% CBN (See FIG. 6), potency by NMR=94.91%, H₂O=0.16% (KF), and 1H NMR confirm the crystals to be pure CBN.

### Example 8

### Synthesis of CBN—250.0 Gram Scale

### Synthesis of Delta8-THC

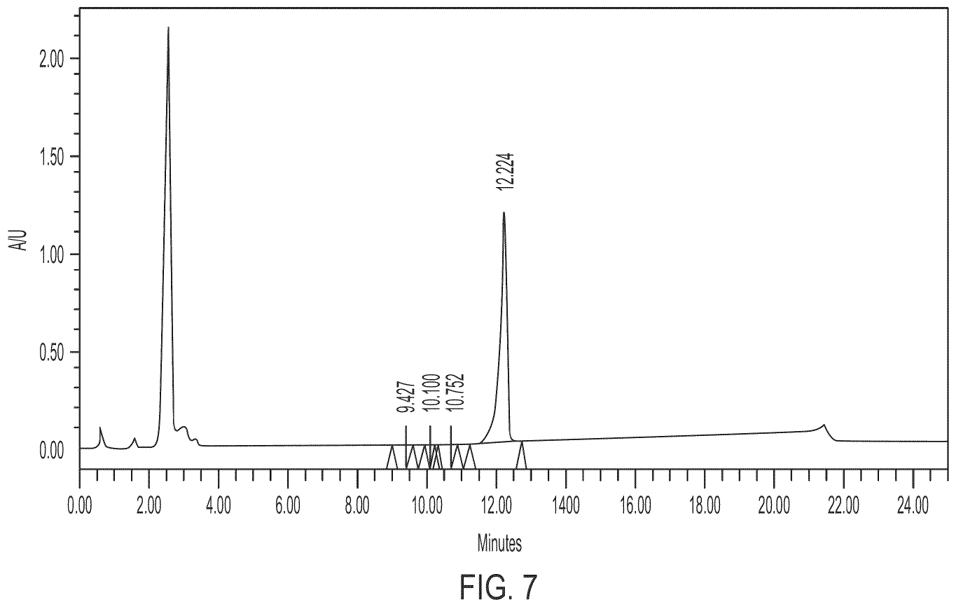

A 30 L reactor equipped with a reflux condenser, overhead mechanical stirring, nitrogen inlet, thermocouple, and connected to a packed tower caustic scrubber containing excess sodium metabisulfite was inerted. CBD (250 g, 0.79 mol, 1.0 equiv) was charged into the reactor followed by toluene (10 L, 40 vol) and pTSA·H₂O (25 g, 0.079 mol, 0.1 equiv.). The reaction mixture was heated to 50° C. and stirred at 1 h after which HPLC indicated >99.9% of CBD to Δ⁸-THC. See FIG. 7.

### Conversion of Delta8-THC to CBN

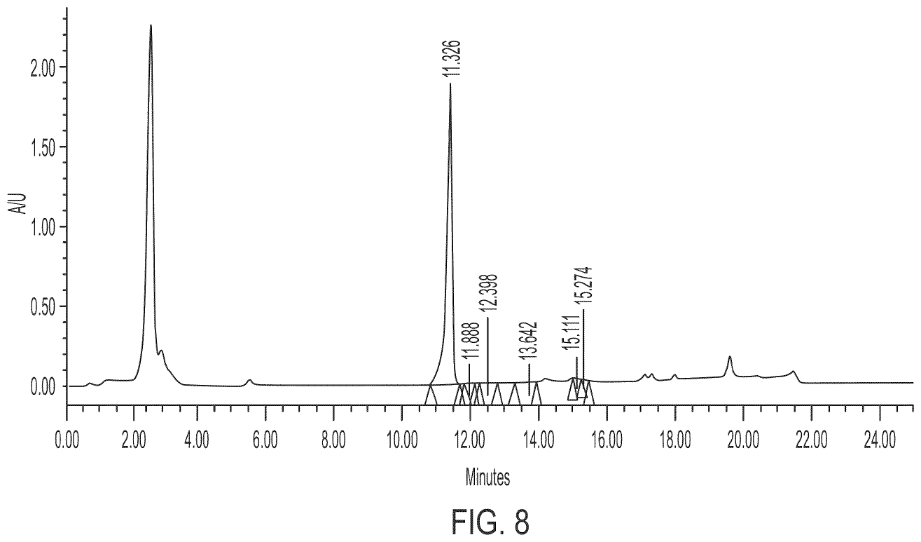

The mixture was cooled to 20° C. and held overnight under nitrogen in a glass carboy while the reactor was cleaned and re-inerted with nitrogen. The inerted reactor was charged with NaHCO₃(274 g, 3.26 mol, 4.1 equiv.) and iodine crystals (444.5 g, 1.75 mol, 2.2 equiv.) followed by the batch solution and some residual colorless solids that had precipitated out of the batch overnight. The batch was heated to 100° C.±3° C., over 4 hours, held for 20 hours at 100° C., and cooled to 52° C. prior to sampling. HPLC analysis indicated 99.7% conversion from Δ⁸-THC to CBN and the peak corresponding to CBN in the chromatogram showed 91.4% CBN. See FIG. 8.

The reaction mixture was cooled to room temperature and removed from the reactor via a 0.2 um in-line filter and held for 2 hours while the reactor was cleaned and re-inerted. The batch, which has a dark reddish color, was washed sequentially with 10% citric acid solution (1.7 L, 1.1 equiv) for 10 min then 10% aq. sodium thiosulfate solution (2×1 L, 2×4 vol) for 10-15 min each time. The batch tested negative for oxidant, iodine, by KI starch paper after the second sodium thiosulfate wash and the wash was colorless. After one final aqueous wash (0.3 L, 1 vol) the organic phase was evaporated on a rotary evaporator to produce 416 g crude CBN as a slightly pinkish dark brown oil.

The material was chromatographed over approximately 4 hours and the fractions collected were held overnight at room temperature to complete HPLC analysis. The selected fractions were concentrated under reduced pressure to produce 281 g of amber oil, which was dissolved in heptanes (840 mL, 3 vol), cooled to approximately 10° C., and seeded with CBN crystals (250 mg, 0.1 wt %) prior to cooling the crystallization mixture to −20° C. The crystallization mixture was held at this temperature, without agitation, for approximately 45 hours.

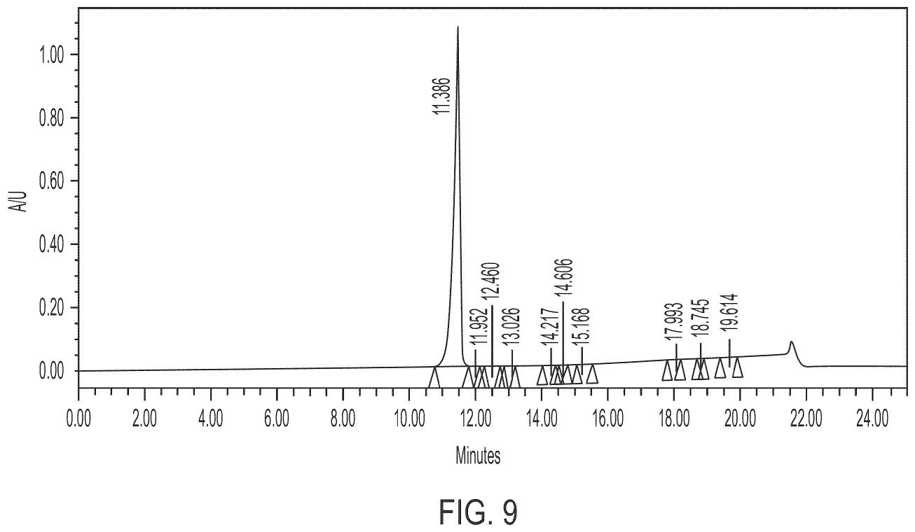

At the end of 45 hours the crystallization mixture was filtered, and the crystals washed with cold heptane. The crystals were dried under high vacuum at room temperature for 3 days to produce a first crop of CBN crystals as an off-white to pale pink solid (108 g, 44% yield, 90.5 wt %, 97.8% purity by the HPLC). See FIG. 9.

### Example 9

### Kilo-Scale Synthesis of CBN

### A. Synthesis of Delta8-THC

A jacketed 750L reactor with ports for introducing solvent, reagents or gas containing 300 L of toluene is fitted with a condenser, a temperature probe, a pressure probe, a mechanical stirrer, a data recorder, thermocouple, and connected to a packed tower caustic scrubber containing excess sodium metabisulfite is inerted. 11.0 Kg of CBD (1.0 eq.) is added to the reactor followed by pTSA·H₂O (1.1 Kg, 0.1 equiv.). Stir the mixture at room temperature using a mechanical stirrer until all the CBD dissolves. The reaction mixture is heated to 50° C. and stirred for 1 h after which HPLC analysis is carried out to monitor conversion of CBD to d8-THC. If conversion is complete, the reaction mixture is cooled to 20° C. and held under an inert atmosphere of nitrogen in a glass carboy while the reactor is cleaned and re-inerted with nitrogen.

### B. Conversion of Delta-8-THC to CBN

The inerted reactor is charged with NaHCO₃(12.1 Kg, 4.1 equiv.) and iodine crystals (19.5 Kg, 2.2 eq.), followed by the addition of the batch solution of delta-8-THC. The reaction mixture is heated to a temperature of 100-110° C., over 4 hours, held for at least 20 hours at 100-110° C., or until HPLC analysis of the reaction indicates greater than 95% conversion of delta-8-THC to CBN.

The crude reaction mixture is cooled to room temperature and removed from the reactor via a 0.2 um in-line filter. The reactor is cleaned, and the crude reaction mixture is then pumped back into the clean reactor, followed by the sequential extraction of the organic layer containing crude CBN with 10% citric acid solution (70 L, 1.1 equiv) for 10 minutes and 10% aq. sodium thiosulfate solution (2×44 L) for 10-15 minutes each time. The reaction mixture is tested for the presence of unreacted, residual oxidant, iodine, by KI starch paper after the second sodium thiosulfate wash. Washing with aqueous sodium thiosulfate is discontinued if the wash is colorless and the test for unreacted iodine using starch paper is negative. After one final aqueous wash (13 L, 1 vol) the organic phase is dried using anhydrous magnesium sulfate and the dry organic solution containing crude CBN is evaporated on a rotary evaporator to produce crude CBN oil which is stored at 2-8° C. until chromatography commences.

The material is chromatographed using silica gel, approximately 8 equivalents with respect to weight of crude CBN and the fractions collected are analyzed by HPLC. The selected fractions containing CBN are concentrated under reduced pressure to produce CBN oil, which is subjected to crystallization. The oil is dissolved in heptanes (3 vol, with respect to the weight of CBN oil), cooled to approximately 10° C., and seeded with CBN crystals (0.1 wt %) prior to cooling the crystallization mixture to −20° C. The crystallization mixture is held at this temperature, without agitation, until pure CBN crystallizes out. The mother liquor is subjected to a second crystallization following concentration.

At the end of crystallization, CBN is filtered, and the CBN crystals washed with cold heptane. The crystals are dried under high vacuum at room temperature for 3-4 days to produce CBN crystals as an off-white to pale pink solid.

### Example 10

### Kilo Scale Synthesis of CBN Using Base

A jacketed 750L reactor (Reactor 1), with ports for introducing solvent, reagents or gas containing 300 L of toluene is fitted with a condenser, a temperature probe, a pressure probe, a mechanical stirrer, and a data recorder. To Reactor 1 add 11.0 Kg of CBD (1.0 eq.). Stir the mixture at room temperature using a mechanical stirrer until all the CBD dissolves. To this solution add 13.1 kg sodium carbonate monohydrate (Na₂CO₃·H₂O, 3.0 equiv.). Thoroughly flush the CBD-Na₂CO₃ solution with nitrogen for about 2-3 hours, while continuously stirring the solution.

After the nitrogen flush is complete, begin heating the solution in Reactor 1 until the temperature of the solution is 110° C., over approximately 30-40 minutes. Maintain the solution in Reactor 1 at 110° C., while continuously stirring the solution under a positive pressure of nitrogen.

To a second jacketed 150L reactor (Reactor 2), with ports for introducing solvent, reagents or gas and equipped with a temperature probe, a pressure probe, a mechanical stirrer, and a data recorder, add 100 L of toluene. To this reactor add 19.6 Kg of iodine (2.2 equiv.). Stir the mixture at room temperature using a mechanical stirrer until all the iodine dissolves. Flush the iodine solution with nitrogen while continuously stirring the solution and then gradually (30-40 min) heat the solution until the temperature is 60° C.

Next, transfer the hot iodine solution into Reactor 1 at a rate that maintains the temperature of the CBD solution in Reactor 1 at 110±5° C. After transfer of the hot iodine solution is complete, maintain the temperature of the reaction mixture in Reactor 1 at 110° C. while continuously stirring the reaction mixture. The progress of the reaction is periodically monitored by HPLC and/or TLC. The reaction is stopped when greater than 90% to 95% of CBD is converted to CBN. At this juncture, stop heating the mixture in Reactor 1 and allow it to cool to room temperature prior to aqueous work-up.

The crude reaction mixture containing unreacted sodium carbonate is filtered and neutralized using an aqueous solution of 10% citric acid. The solution of citric acid is made in a separate quench tank by dissolving 20.1 Kg of citric acid in 200L of water at room temperature. Neutralization of unreacted sodium carbonate is carried out by gradually pumping the citric acid solution from the quench tank into Reactor 1. Since the neutralization of unreacted sodium carbonate by citric acid releases carbon dioxide gas, this process is carried out in a properly ventilated walk-in hood. After complete addition of aqueous citric acid to Reactor 1, the organic-aqueous mixture in Reactor 1 is mechanically stirred (˜30-45 minutes), following which stirring is stopped and the organic layer containing crude CBN is separated from the aqueous layer.

The pH of the separated aqueous layer is measured, and a second neutralization is carried out if the pH of the first aqueous layer is greater than 3.5-4.0. Following neutralization, the organic solution containing crude CBN, unreacted iodine and unreacted CBD is extracted with 80 liters of an aqueous solution of 10% sodium sulfite (Na₂SO₃, weight/volume). The aqueous solution of sodium sulfite is prepared separately in an Extraction tank by dissolving 8.0 kg sodium sulfite in 80L water at room temperature and is pumped into Reactor 1. Alternatively, unreacted iodine is removed by extracting the crude reaction mixture with 80 liters of an aqueous solution of 10% sodium thiosulfate pentahydrate (Na₂S₂O₃·5H₂O, weight/volume). Extraction of the crude reaction mixture with an aqueous solution of 10% (weight/volume) sodium sulfite or an aqueous solution of 10% (weight/volume) sodium thiosulfate pentahydrate is carried out twice. The organic layer containing crude CBN, and unreacted CBD is washed with water, about 80L twice, following which the aqueous layer is separated from the organic layer.

Reactor 1 containing the organic solution of crude CBN and unreacted CBD is then fitted with a distillation column and condenser. The volume of the crude reaction mixture is reduced to be in a range from about 50L-75L. The concentrated reaction mixture is cooled to room temperature and purified to obtain CBN.

### Example 11

### Kilo Scale Synthesis of CBNV Using Base

The synthesis of CBNV is carried out using a protocol similar to the one described above for CBN. The synthesis of CBNV uses CBDV as starting material. CBDV is synthesized using bio-catalysis as described in U.S. Pat. No. 10,336,978 and incorporated herein by reference in its entirety. Alternatively, CBDV from botanical sources or chemically synthesized CBDV can be used for the synthesis of CBNV. Briefly, the oxidation of CBDV to CBNV is carried out as follows.

Add 30 liters of toluene to a jacketed 100L reactor (Reactor 1), with ports for introducing solvent, reagents or gas and fitted with a condenser, a temperature probe, a pressure probe, a mechanical stirrer, and a data recorder. To Reactor 1 add 1.0 Kg of CBDV (1.0 equiv., MW 286.41 g/mol, 3.49 moles). Stir the reaction mixture at room temperature using a mechanical stirrer until all the CBDV dissolves. To this solution add 1.298 kg sodium carbonate monohydrate (Na₂CO₃·H₂O, 3.0 equiv., MW 124.0 g/mol, 10.47 moles). Flush the CBDV-Na₂CO₃ solution with nitrogen for about 2 hours, while continuously stirring the solution.

After the nitrogen flush is complete, gradually begin heating the solution until the temperature of the solution in Reactor 1 is 110° C., over approximately 30-40 minutes. Maintain the solution in Reactor 1 at 110° C., with continuous stirring and under a positive pressure of nitrogen.

To a second jacketed 20L reactor (Reactor 2), with ports for introducing solvent, reagents or gas and equipped with a temperature probe, a pressure probe, a mechanical stirrer, and a data recorder, add 10.0 L of toluene. To this reactor add 1.948 Kg of iodine (MW 253.8 g/mol, 7.7 moles, 2.2 equiv.). Stir the mixture at room temperature using a mechanical stirrer until the iodine dissolves. Flush the solution with nitrogen while stirring it and then gradually (30)-40 min) heat the solution until the temperature of the solution is 60° C.

Pump the hot iodine solution from Reactor 2 into Reactor 1 at a rate that maintains the temperature of the CBDV-Na₂CO₃ solution in Reactor 1 at 110±5° C. After the transfer is completed, continue stirring the reaction mixture in Reactor 1 while maintaining the temperature of the solution at 110° C. When greater than 90%-95% of CBDV is converted to CBNV, as judged by HPLC, stop heating the mixture in Reactor 1 and allow it to cool to room temperature prior to aqueous work-up.

The crude reaction mixture containing unreacted iodine and sodium carbonate is filtered and neutralized using an aqueous solution of 10% citric acid (1×50L). Neutralization is carried out by mechanically stirring the citric acid-toluene solution in Reactor 1 for 30-45 minutes, or at least until evolution of carbon dioxide gas ceases.

After evolution of gas ceases, mechanical stirring is stopped and the organic and aqueous layers in Reactor 1 are allowed to separate. The pH of the separated aqueous layer is measured and a second extraction with 10% citric acid is carried out if the pH of the aqueous layer from the first extraction is greater than 3.5-4.0. The neutralized organic layer containing crude CBN is then extracted with 10% aqueous sodium sulfite (Na₂SO₃, weight/volume: ˜30-40 liters) to remove unreacted iodine. Alternatively, unreacted iodine can be removed by extraction of the crude reaction mixture with an aqueous solution of 10% sodium thiosulfate pentahydrate (Na₂S₂O₃·5H₂O, weight/volume: ˜30-40 liters). Finally, the organic layer containing crude CBNV and some unreacted CBDV is extracted with distilled water, about 40L twice.

Reactor 1 containing a solution of crude CBNV and unreacted CBDV is then fitted with a distillation column and condenser. The crude reaction mixture is concentrated by reducing the volume of toluene (organic solvent) to about 4.0L-5.0L. The concentrated mixture of crude CBNV is then cooled to room temperature and CBNV is purified as described above for CBN.

While the compositions and methods of this invention have been described in terms of the foregoing illustrative embodiments, it will be apparent to those of skill in the art that variations, changes, modifications, and alterations may be applied to the composition, methods, and in the steps or in the sequence of steps of the methods described herein, without departing from the true concept, spirit, and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope, and concept of the invention as defined by the appended claims.

### Enumerated Embodiments

The following enumerated embodiments are provided, the numbering of which is not to be construed as designating levels of importance.

Embodiment 1 provides a method for synthesizing cannabinol (CBN), comprising:
(a) contacting cannabidiol (CBD) with a solvent and a base and heating the resulting first mixture;
(b) contacting an oxidant with a solvent and heating the resulting second mixture; and
(c) adding the heated mixture of step (b) to the heated mixture of step (a).

Embodiment 2 provides the method of embodiment 1, wherein the solvent for step (a) and step (b) is selected from the group consisting of hexane, heptane, xylene, ortho-xylene, meta-xylene, para-xylene, and toluene.

Embodiment 3 the method of embodiment 1 or 2, wherein the first mixture is heated to a temperature in the range from 110° C. to 130° C.

Embodiment 4 provides the method of embodiment 1 or 2, wherein the second mixture is heated to a temperature in the range from 60° C. to 90° C.

Embodiment 5 provides the method of embodiment 1, wherein the oxidant is selected from the group consisting of bromine, iodine, DDQ, and DDQ/para-toluenesulfonic (p-TsOH) acid.

Embodiment 6 provides the method of embodiment 1, wherein the base is an organic base or an inorganic base.

Embodiment 7 provides the method of embodiment 6, wherein the base is an inorganic base selected from the group consisting of sodium carbonate, potassium carbonate, sodium bicarbonate, potassium bicarbonate, sodium hydrogen phosphate, and potassium hydrogen phosphate.

Embodiment 8 provides the method of any one of embodiments 1-7, further comprising purifying the cannabinol (CBN).

Embodiment 9 provides a method for synthesizing cannabinovarin (CBNV), comprising:
(a) contacting cannabidivarin (CBDV) with a solvent and a base and heating the resulting first mixture;
(b) contacting an oxidant with a solvent and heating the resulting second mixture; and
(c) adding the heated mixture of step (b) to the heated mixture of step (a).

Embodiment 10 provides the method of embodiment 9, wherein the solvent for step (a) and step (b) is selected from the group consisting of hexane, heptane, xylene, ortho-xylene, meta-xylene, para-xylene, and toluene.

Embodiment 11 provides the method of embodiment 9 or 10, wherein the first mixture is heated to a temperature in the range from 110° C. to 130° C.

Embodiment 12 provides the method of embodiment 9 or 10, wherein the second mixture is heated to a temperature in the range from 60° C. to 90° C.

Embodiment 13 provides the method of embodiment 9, wherein the oxidant is selected from the group consisting of bromine, iodine, DDQ, and DDQ/p-toluenesulfonic acid.

Embodiment 14 provides the method of embodiment 9, wherein the base is an organic base or an inorganic base.

Embodiment 15 provides the method of embodiment 14, wherein the base is an inorganic base selected from the group consisting of sodium carbonate, potassium carbonate, sodium bicarbonate, potassium bicarbonate, sodium hydrogen phosphate, and potassium hydrogen phosphate.

Embodiment 16 provides the method of any one of embodiments 9-15, further comprising purifying the cannabinovarin (CBNV).

Embodiment 17 provides a method for synthesizing cannabinol (CBN) or cannabinovarin (CBNV), comprising:
(a) contacting a compound of Formula 1 with a solvent and an acid to form a first mixture:

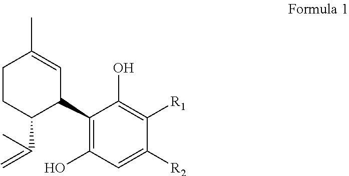

(b) heating the resulting first mixture from (a) to form a compound according to Formula 2; and

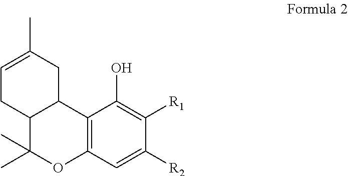

(c) contacting an oxidant with the mixture of step (b) and heating the resultant second mixture to form CBN;
   wherein,
R₁ in Formula 1 and Formula 2 is —H and R₂ in Formula 1 and Formula 2 is propyl or pentyl.

Embodiment 18 provides the method of embodiment 17, wherein the first mixture is heated to 50° C.

Embodiment 19 provides the method of embodiment 17, wherein the second mixture is heated to 110° C.

Embodiment 20 provides the method of embodiment 17, wherein the acid is selected from the group consisting of hydrochloric acid, para-toluene sulfonic acid (p-TSA), citric acid, aluminum trichloride, boron trifluoride etherate, magnesium sulfate, Amberlyst IR-120 Plus (H), Amberlyst IR-120 Plus, Amberlyst A-15, Amberlyst IRP-69, and Amberlyst 1200(H).

### Other Embodiments

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

I/We claim:

1. A method for synthesizing cannabinol (CBN), comprising:
(a) contacting cannabidiol (CBD) with a solvent and a base and heating the resulting first mixture;
(b) contacting an oxidant with a solvent and heating the resulting second mixture; and
(c) adding the heated mixture of step (b) to the heated mixture of step (a).

2. The method of claim 1, wherein the solvent for step (a) and step (b) is selected from the group consisting of hexane, heptane, xylene, ortho-xylene, meta-xylene, para-xylene, and toluene.

3. The method of claim 1, wherein the first mixture is heated to a temperature in the range from 110° C. to 130° C.

4. The method of claim 1, wherein the second mixture is heated to a temperature in the range from 60° C. to 90° C.

5. The method of claim 1, wherein the oxidant is selected from the group consisting of bromine, iodine, DDQ, and DDQ/para-toluenesulfonic (p-TsOH) acid.

6. The method of claim 1, wherein the base is an organic base or an inorganic base.

7. The method of claim 6, wherein the base is an inorganic base selected from the group consisting of sodium carbonate, potassium carbonate, sodium bicarbonate, potassium bicarbonate, sodium hydrogen phosphate, and potassium hydrogen phosphate.

8. The method of claim 1, further comprising purifying the cannabinol (CBN).

9. A method for synthesizing cannabinovarin (CBNV), comprising:
(a) contacting cannabidivarin (CBDV) with a solvent and a base and heating the resulting first mixture;
(b) contacting an oxidant with a solvent and heating the resulting second mixture; and
(c) adding the heated mixture of step (b) to the heated mixture of step (a).

10. The method of claim 9, wherein the solvent for step (a) and step (b) is selected from the group consisting of hexane, heptane, xylene, ortho-xylene, meta-xylene, para-xylene, and toluene.

11. The method of claim 9, wherein the first mixture is heated to a temperature in the range from 110° C. to 130° C.

12. The method of claim 9, wherein the second mixture is heated to a temperature in the range from 60° C. to 90° C.

13. The method of claim 9, wherein the oxidant is selected from the group consisting of bromine, iodine, DDQ, and DDQ/p-toluenesulfonic acid.

14. The method of claim 9, wherein the base is an organic base or an inorganic base.

15. The method of claim 14, wherein the base is an inorganic base selected from the group consisting of sodium carbonate, potassium carbonate, sodium bicarbonate, potassium bicarbonate, sodium hydrogen phosphate, and potassium hydrogen phosphate.

16. The method of claim 9, further comprising purifying the cannabinovarin (CBNV).

17. A method for synthesizing cannabinol (CBN) or cannabinovarin (CBNV), comprising:
(a) contacting a compound of Formula 1 with a solvent and an acid to form a first mixture; 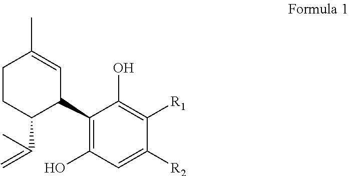
(b) heating the resulting first mixture from (a) to form a compound according to Formula 2; and 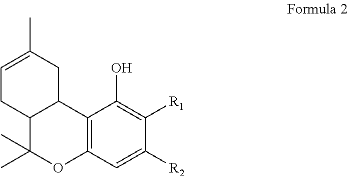
(c) contacting an oxidant with the mixture of step (b) and heating the resultant second mixture to form CBN;
wherein,
R₁ in Formula 1 and Formula 2 is —H and R₂ in Formula 1 and Formula 2 is propyl or pentyl.

18. The method of claim 17, wherein the first mixture is heated to 50° C.

19. The method of claim 17, wherein the second mixture is heated to 110° C.

20. The method of claim 17, wherein the acid is selected from the group consisting of hydrochloric acid, para-toluene sulfonic acid (p-TSA), citric acid, aluminum trichloride, boron trifluoride etherate, magnesium sulfate, Amberlyst IR-120 Plus (H), Amberlyst IR-120 Plus, Amberlyst A-15, Amberlyst IRP-69, and Amberlyst 1200(H).
